(19) United States
(12) Patent Application Publication
(10) Pub. No.: US 2026/0080541 A1
(43) Pub. Date: Mar. 19, 2026

(54) METHODS FOR PROVIDING AN ITEM OF COMPARISON INFORMATION FOR A MEDICAL IMAGING APPARATUS

(71) Applicant: Siemens Healthineers AG, Forchheim (DE)

(72) Inventors: David GRODZKI, Erlangen (DE); Till HUELNHAGEN, Erlangen (DE); Rainer SCHNEIDER, Hoechstadt (DE)

(73) Assignee: Siemens Healthineers AG, Forchheim (DE)

(21) Appl. No.: 19/332,114

(22) Filed: Sep. 18, 2025

(30) Foreign Application Priority Data
Sep. 19, 2024 (DE) 10 2024 209 002.6

(51) Int. Cl. G06T 7/00; A61B 5/055; G06T 7/60; G06T 11/00

(52) U.S. Cl. CPC G06T 7/0016; G06T 7/60; G06T 12/20; G06T 12/30; A61B 5/055; G06T 2200/24; G06T 2207/10081; G06T 2207/10088; G06T 2207/10104; G06T 2207/20076; G06T 2207/20081; G06T 2207/20084; G06T 2207/30016; G06T 2207/30061; G06T 2207/30068; G06T 2210/41

(57) ABSTRACT

One or more example embodiments relates to a method for providing an item of comparison information based on an item of output information for a medical imaging apparatus. In addition, one or more example embodiments relates to a computing unit, a medical imaging apparatus, a computer program product and a computer storage medium. The computer-implemented method for providing an item of comparison information based on an item of output information for a medical imaging apparatus, comprises generating expected measurement data via a trained function based on the item of output information, providing measurement data of a medical imaging examination based on the items of output information, ascertaining an item of comparison information based on the expected measurement data and the measurement data, and providing the item of comparison information.

### CROSS-REFERENCE TO RELATED APPLICATION(S)

The present application claims priority under 35 U.S.C. § 119 to German Patent Application No. 10 2024 209 002.6, filed Sep. 19, 2024, the entire contents of which are incorporated herein by reference.

### FIELD

One or more example embodiments relates to a method for providing an item of comparison information on the basis of an item of output information for a medical imaging apparatus. In addition, one or more example embodiments relates to a computing unit, a medical imaging apparatus, a computer program product and a computer storage medium.

### RELATED ART

Medical imaging, in particular magnetic resonance (MR) imaging, enables the diagnosis and therapy of various diseases or conditions. In this connection, measurement data is typically captured which reflects a spatial and/or temporal distribution of a physical parameter of a tissues of a region of the body of a patient. Images can be reconstructed from the captured measurement data, and these supply anatomical or functional items of information about the examined region of the body.

However, the quality and validity of the measurement data and the images reconstructed from it depends on a large number of factors which relate to the medical imaging apparatus as well as the patient and the examination situation. Thus, for example, system errors, unexpected items of information, artifacts, movements, anatomical deviations or pathological changes can influence or impair the measurement data. A diagnosis can be made more difficult or even impossible as a result. It es therefore advantageous to identify deviations or anomalies in the measurement data early and to adjust the imaging accordingly.

The prior art offers various solutions for identifying deviations or anomalies in the measurement data or the reconstructed images. Methods, for example, are known for identifying artifacts in MR images, for example based on a statistical analysis of pixel values. In addition, methods, for example, are known for identifying specific system errors in MR images. However, these solutions are usually designed for specific types of deviations anomalies and cannot generally be applied to all possible situations. In addition, they often require a prior reconstruction of the images from the measurement data, and this means additional time and computing outlay.

### SUMMARY

One or more example embodiments provide an improved method for providing an item of information on the basis of an item of output information for a medical imaging apparatus, which method enables early and comprehensive identification of deviations or anomalies in the measurement data of a medical imaging examination.

The is achieved by the subject matter of the independent claims. Advantageous embodiments can be found, inter alia, in the features of the dependent claims.

### BRIEF DESCRIPTION OF THE DRAWINGS

Advantages, features and details can be found in example embodiments described below as well as on the basis of the drawings. Mutually corresponding parts are provided with identical reference numerals in all figures. Modifications mentioned in this connection can be combined with one another respectively in order to embody new embodiments.

In the drawings:

FIG. 1 shows a flowchart of a method for providing an item of comparison information on the basis of an item of output information for a medical imaging apparatus according to a preferred embodiment,

FIG. 2 shows a data flow diagram of a method for providing an item of comparison information on the basis of an item of output information according to one embodiment,

FIG. 3 shows a flowchart of a method for providing an item of comparison information on the basis of an item of output information, comprising optional method steps according to one embodiment,

FIG. 4 shows a schematic representation of an embodiment of a computing unit for providing an item of comparison information on the basis of items of output information,

FIG. 5 shows a schematic representation of the trained function incorporated by the method according to an embodiment, and

FIG. 6 shows a schematic representation of an embodiment of the medical imaging apparatus as a magnetic resonance apparatus.

### DETAILED DESCRIPTION

Accordingly, a computer-implemented method is proposed according to one or more example embodiments for providing an item of comparison information on the basis of an item of output information for a medical imaging apparatus, comprising the following steps:
generating expected measurement data via a trained function on the basis of the item of output information
providing measurement data of a medical imaging examination on the basis of the items of output information
ascertaining an item of comparison information on the basis of the expected measurement data and the measurement data,
providing the item of comparison information.

The item of output information comprises at least one item of patient information and/or at least one item of imaging information.

The item of comparison information can comprise, in particular, an item of discrepancy information and/or an item of correlation information of the expected measurement data with the measurement data and/or a threshold value. In particular, the item of comparison information can comprise items of information about deviations, errors and/or peculiarities, which are possible and/or have occurred, in the medical imaging examination mapped by the measurement data compared with a virtual imaging examination mapped by the expected measurement data.

The item of output information comprises, in particular, information which enables implementation and/or execution of a medical imaging examination via the medical imaging apparatus. The item of output information can be captured, provided, generated and/or received, in particular before, during and/or after the medical imaging examination, in particular by a computing unit, in particular of the medical imaging apparatus. The item of output information can comprise, in particular, one and/or more item(s) of information from one or more source(s) of information, for example an electronic patient file and/or a database and/or the medical imaging apparatus. The item of output information can comprise, in particular, two items of partial information, in particular at least one item of patient information and at least one item of imaging information. The items of partial information can be provided, in particular together, as an item of output information or as items of partial information. In particular, the items of output information can be the input data for the generation of the expected measurement data and/or the input data for ascertaining the measurement data via imaging.

The item of patient information can comprise, in particular, patient data, in particular from an electronic patient file, examination information, in particular pre-scans, items of information of a medical diagnosis, and/or preliminary examination information, in particular image data and/or parameters from preliminary examinations. In other words, the items of patient information can comprise, for example, items of information about the patient, such as a height, weight, age, a medical history of the patient, a health status, diagnosis and/or a treatment goal of the patient.

The item of imaging information can comprise, in particular, items of information for controlling a medical imaging apparatus and/or imaging parameters, in particular a measurement protocol for carrying out a medical imaging examination. The item of imaging information can comprise, in particular, items of information relating to carrying out a magnetic resonance examination via a magnetic resonance apparatus. In particular, the item of imaging information can comprise a measurement protocol and/or a measuring sequence. For example, the imaging parameters can comprise a slice interval, slice number, slice thickness, a turbo factor and/or a flip angle. In other words, the items of imaging information can comprise, in particular, items of information about a type of examination and/or imaging apparatus, parameters, values, setting information and/or a protocol of the medical imaging examination.

The medical imaging apparatus is conventionally embodied for capturing medical and/or diagnostic image data of a patient. In particular, an imaging examination can be carried out via the imaging apparatus on the basis of a measurement protocol and/or imaging protocol. In particular, the medical imaging apparatus can comprise a magnetic resonance apparatus, a computed tomography apparatus and/or positron emission tomography apparatus (PET).

The expected measurement data can comprise, in particular, synthetic measurement data provided via the trained function on the basis of the item of output information, raw data, image data and/or K-space measurement data. In particular the expected measurement data can be provided on the basis of a simulated medical imaging examination via the trained function on the basis of the item of output information. The expected measurement data can comprise, in particular, simulated, virtual, synthetic and/or artificially generated data, in particular measurement data, and/or items of information of an (in particular, simulated, virtual, synthetic) medical imaging examination.

The measurement data can comprise, in particular, data and/or items of information which are generated during a medical imaging examination via the medical imaging apparatus, in particular on the basis of the items of output information. The measurement data can include, for example, items of information about the intensity and/or phase of magnetic resonance rays or X-rays which are, for example, reflected, absorbed or emitted by a patient. The measurement data can comprise, in particular, data in different data formats, for example raw data, K-space data or image space data. The measurement data can be used, in particular, as input data for reconstructing images. The measurement data can comprise, in particular, items of information, in particular reconstructed image data, about the anatomical and/or functional properties of the examined patient. The measurement data can comprise, in particular, signals captured during a medical imaging examination.

Generating the expected measurement data via a trained function on the basis of the item of output information comprises, in particular, applying the trained function to the item of output information, in particular the item of imaging information and/or the item of patient information. In particular, generating the expected measurement data can comprise simulating a medical imaging examination. In other words, the trained function preferably generates expected measurement data which is comparable with the measurement data of the real medical imaging examination and/or resembles it. In particular, generating the expected measurement data can comprise generating a virtual and/or artificial magnetic resonance response on the basis of the item of imaging information and/or the item of patient information. In particular, generating the expected measurement data can comprise converting the virtual and/or artificial magnetic resonance response in expected measurement data. In other words, in particular a raw dataset can thus be generated as the expected measurement data, which is suitable for comparison with a measured raw dataset of the medical imaging examination.

Providing the measurement data can comprise, in particular, capturing, in particular via the medical imaging apparatus during a medical imaging examination, and/or storing the measurement data, in particular on a storage medium, and/or accessing stored measurement data.

Ascertaining an item of comparison information on the basis of the expected measurement data and the measurement data comprises, in particular, determining and/or calculating a difference, a deviation, a discrepancy, a match or a correlation between the expected measurement data and the measurement data. In particular, the method comprises a method step of determining a similarity measure with a similarity function by comparing the synthetic image data, in particular the expected measurement data, and with medical image data, in particular the measurement data. The method can, in particular, comprise moreover a method step of adjusting at least one parameter (of the trainable/trained function) by optimizing the similarity function of the trainable function based on the similarity measure. The method step of ascertaining the item of comparison information can be executed, in particular, in a processor, a memory, a display unit or a combination of these. The method step of ascertaining can be carried out, in particular, during or after the medical imaging examination and/or the image reconstruction.

Providing the item of comparison information, in particular to a user and/or the medical imaging apparatus, comprises, in particular, displaying, transmitting, storing or processing the item of comparison information. The item of comparison information can be displayed to a user, for example, via a display unit of the medical imaging apparatus. The item of comparison information can be stored, in particular, in a database, a computing unit and/or control unit, be transmitted to them and/or be processed further via them.

The trained function (also referred to as a trainable function) can be, for example, a neural network, which was trained on the basis of a dataset of real measurement data and associated items of output information in order to generate a virtual and/or artificial magnetic resonance response and transfer it in an expected measurement dataset. Synthetic image data can be generated by applying the trained function to the items of output information. The method can additionally comprise a method step of providing the trainable function. In general, a trainable function mimics cognitive functions which humans connect with human thinking. In particular, the trainable function can adapt to new circumstances as well as identify and extrapolate patterns by way of training based on training data.

In general, parameters of a trainable function can be adjusted via training. In particular, supervised training, semi-supervised training, unsupervised training, reinforcement learning and/or active learning can be used for this. Furthermore, representation learning (an alternative term is feature learning) can be used. In particular, the parameters of the trainable functions can be adjusted by a plurality of training steps.

In particular, a trainable function can comprise a neural network, a support vector machine, a decision tree and/or a Bayesian network, and/or the trainable function can be based on k-means clustering, Q-learning, genetic algorithms and/or association rules. In particular, a trainable function can comprise a combination of a plurality of uncorrelated decision trees or an ensemble of decision trees (random forest). In particular, the trainable function can be determined via XGBoosting (eXtreme Gradient Boosting). In particular, a neural network can be a deep neural network, a convolutional neural network or a convolutional deep neural network. Furthermore, a neural network can be an adversarial network, deep adversarial network and/or a generative adversarial network. In particular, a neural network can be a recurrent neural network. In particular, a recurrent neural network can be a network with long-short-term-memory (LSTM), in particular a Gated Recurrent Unit (GRU). In particular, a trainable function can comprise a combination of the described approaches. In particular, the approaches described here are cited for a trainable function network architecture of the trainable function.

The method can enable early and comprehensive identification of deviations and/or anomalies in the measurement data of a medical imaging examination and thereby improve the quality and accuracy of the measurement data and/or the reconstructed images. In particular, the item of output information, a measurement protocol and/or an item of imaging information, the trained function and/or the image reconstruction method can be adjusted by the proposed method, or, in particular, the provided item of comparison information, to the specific requirements of the user and/or the medical imaging apparatus and/or examination. The proposed method can thereby increase the efficiency and reliability of the medical imaging (examination) in that it can enable automatic and continuous quality control and optimization by way of identification of potential errors, artifacts or deviations. The item of comparison information can additionally enable the user to assess and optimize the quality and/or accuracy of the measurement data and/or the reconstructed images. Providing the item of comparison information can enable this, in particular in real time, with a delay or in a deferred manner.

According to one or more example embodiments, generating the expected measurement data comprises executing an image reconstruction method in reverse order. Providing the measurement data of the medical imaging examination comprises executing the image reconstruction method in forward order.

In particular, the image reconstruction method can be executed in reverse order via the trained function. In particular, the image reconstruction method can be executed in reverse order on the basis of the item of output information. In particular, the image reconstruction method can be executed in forward order via a, in particular second, trained function and/or on the basis of the measurement data, in particular measured raw data. In particular, the image reconstruction method can comprise the same steps in forward and reverse order. In particular, the image reconstruction method executed in forward and reverse order can comprise different steps.

The image reconstruction method enables, in particular, generating and/or reconstructing an image and/or image data from a measured raw dataset and/or captured information of a medical imaging apparatus during an imaging examination. For this, the image reconstruction method can comprise, in particular, one and/or more method(s), process(es) and/or algorithm(s) which can be implemented, in particular, via one or more trained function(s). The image reconstruction method can comprise, for example, the use (and/or an application) of a, in particular first, trained function which was trained on the basis of a dataset of real measurement data (and/or raw data) and corresponding associated items of output information to generate a virtual, synthetic and/or artificial magnetic resonance response on the basis of items of output information. The, in particular first, trained function can be, for example, a neural network which can be executed in forward and/or reverse order. The image reconstruction method can comprise, in particular, a second trained function which is embodied for generating a medical image and/or image data from ascertained raw data and/or measurement data of the medical imaging apparatus.

The image reconstruction method can additionally comprise carrying out a Fourier transform, via which it is possible to generate, in particular from the measured raw data, in particular measurement data, magnitude or phase images. The Fourier transform can be combined, in particular, with various filters incorporated by the image reconstruction method, such as high-pass, low-pass or smoothing filters, in order, for example, to modify a signal-to-noise ratio or a spatial resolution of the reconstructed image data. The image reconstruction method can additionally comprise, in particular, applying volume representation and/or post-processing methods, such as Maximum Intensity Projection (MIP), Multiplanar reconstruction (MPR) or Curved Planar Reconstruction (CPR). In particular, the volume representation methods can comprise calculating items of image information from the 3D or multi-layer data in any orientation and/or along an anatomical structure. The image reconstruction method can additionally comprise, in particular, one or more method step(s) of an image evaluation, for example an image subtraction, averaging, rotation, inversion and the like. It is possible to reconstruct phase images, in particular from measured raw data, via the image reconstruction method. In particular moved spin ensembles may be differentiated from stationary tissue in phase images, in particular a magnetic resonance examination, since stationary spins have an identical phase, but moved spins have differing phases, dependent on their speed respectively.

The image reconstruction method can additionally comprise, in particular, applying a filter, in particular a spatial filter and/or image data filter. The spatial filter can be, in particular, a parameter for smoothing images, which can bring about an increase in the signal-to-noise ratio while simultaneously reducing the spatial resolution. The image data filter can comprise, in particular, a filter for filtering a noise-distorted (magnetic resonance) image at different strengths (strong, medium, weak). In particular, the image data filter can comprise high-pass and low-pass filters with different edges in the characteristic curves.

Executing the image reconstruction method in forward order can comprise, in particular, successively executing predefined sub-steps and/or methods in a predetermined order. The forward execution of the image reconstruction method can likewise be referred to as executing the image reconstruction method in the primary order. Executing the image reconstruction method in reverse order can comprise, in particular, executing the sub-steps and/or methods in an order which is the opposite of the forward order.

Executing the image reconstruction method in reverse order enables a simplified provision of the expected measurement data. This can enable early identification of errors or inconsistencies in the measurement data via a comparison, so improvement the image quality of the reconstructed images can be enabled in turn. In particular, the reverse reconstruction can allow the optimum parameters and settings of the imaging to be identified and adjusted. Potential problems and artifacts, which can occur in the forward reconstruction, can be analyzed and corrected better by the reverse order. The method can contribute to optimizing the time and resources which are required for the image reconstruction in that unnecessary steps can be eliminated and only the relevant data can be used. This aspect of the method can enable increased flexibility when adjusting to different imaging requirements and conditions since a dynamic adjustment of the reconstruction parameters is possible.

According to one or more example embodiments, ascertaining an item of comparison information comprises comparing the expected measurement data with the measurement data. The item of comparison information comprises an item of outcome information of the comparison.

The item of comparison information can comprise, in particular, an item of outcome information of a, in particular value-based, comparison between the measurement data and the expected measurement data. For example, the item of comparison information can comprise an item of discrepancy information, an item of correlation information, an item of deviation information, an item of quality information and/or an item of evaluation information. In other words, the item of comparison information can comprise, in particular, a quantitative and/or qualitative item of information about a match and/or deviation of the measurement data with/from the expected measurement data. In particular the item of output information can be validated, verified, optimized, corrected, modified and/or supplemented, in particular via a computing unit and/or a processor, via and/or on the basis of the item of comparison information. In particular, the item of comparison information can enable the adjustment and/or updating of the item of output information, in particular the item of imaging information, in particular of a measurement protocol and/or imaging parameters. The item of comparison information can be provided, in particular, in the form of a number, a graph, a histogram, a table, a text, a symbol, a signal, a color, a scale, or the like and/or any combination.

The measurement data and/or the expected measurement data can be converted, in particular, into a data format, for example DICOM, NIfTI or HDF5, to enable a comparison of the measurement data and/or the expected measurement data. The measurement data and the expected measurement data can be transferred, in particular, to a comparison unit which can comprise a computing module, a processor or the like for carrying out the comparison. The comparison unit can be embodied to execute a plurality of algorithms and/or methods in order to compare the measurement data and the expected measurement data. A method can comprise, for example, a pixel-by-pixel or voxel-by-voxel discrepancy test, a correlation test, a Chi-squared test, a t-test, an F-test or the like. Comparing the measurement data with the expected measurement data can comprise, in particular, a Fourier transform or a wavelet transform. In particular, comparing the measurement data with the expected measurement data can comprise a transformation of the data into a shared data space. The data points of the measurement data and the expected measurement data can be compared with each other, in particular, in this data space, in particular by a comparison unit, for example by calculating a discrepancy, a correlation, a deviation, a quality measure or an assessment measure. In particular, the measurement data and the expected measurement data can also be compared directly and/or in the image space, in particular by the comparison unit, for example by applying an image registration, a segmentation, a classification, a pattern recognition, a feature extraction or a feature analysis.

The item of comparison information enables a quick and precise checking of whether the measurement data corresponds with the ascertained expected measurement data and thereby whether the imaging apparatus is functioning properly. The item of comparison information can supply indications of potential sources of error, interference, artifacts or other deviations which can impair the accuracy, reliability or reproducibility of the measurement data or the imaging examination. The item of comparison information can serve as feedback information for an optimization, correction, modification or supplementation of the item of output information, in particular of a measurement protocol and of imaging parameters of the item of imaging information of the item of output information, in order to improve or adjust the quality or output of the imaging apparatus, or imaging examination. The item of comparison information can be used as the basis and/or as basic information for quality control or quality assessment and/or a validation, verification or calibration of the imaging apparatus or the measurement data.

According to one or more example embodiments, ascertaining an item of comparison information comprises comparing the expected measurement data and/or the measurement data with at least one reference value.

In particular, the reference value comprises a threshold value.

The reference value, in particular threshold value, can comprise, in particular, a specified numerical value. The reference value, in particular threshold value, can be derived, for example, via a statistical analysis of a representative sample of measurement data. The reference value, in particular threshold value, can, in particular, also be an empirically or a theoretically determined value which is based on specifications and/or expectations of the imaging apparatus and/or a user. The reference value or threshold value can be defined, for example, on the basis of standard data, industry standards, clinical requirements or user preferences. The reference value or threshold value can be different, for example, for each parameter, each sequence or each measuring method.

In particular if the measurement data and/or the item of comparison information overshoots or undershoots a reference value, this can be an indication of an error, an interference, an artifact or another deviation, in particular of the imaging apparatus. The use of threshold value can enable a decision, in particular by a user, as to whether the measured values lie within an acceptable range. The simplicity and efficiency which a comparison offers via a threshold value is additionally advantageous. Compared with more complex comparison methods, the use of a threshold value is easy to implement, to modify and to check. This facilitates the automation of the comparison in addition to allowing fast and uncomplicated adjustment of the threshold values in order to map different requirements. In addition, in particular a reference and/or threshold value can contribute to an improvement in the image quality in that it serves as a criterion for the acceptance or rejection of measurement data in method sequences. Finally, the use of threshold values can enable consistent and reproducible evaluation which guarantees the comparability and traceability of measurements across different instants and conditions.

According to one or more example embodiments, the method comprises ascertaining and/or updating a reference value, in particular threshold value, comprising one or more of the following step(s):
providing, in particular initially and/or periodically during a medical imaging examination, a representative sample from the expected measurement data and/or measurement data
applying, in particular initially and/or periodically during a medical imaging examination, a statistical evaluation of the expected measurement data and/or the measurement data to derive an initial reference value, in particular threshold value, in particular on the basis of the representative sample,
comparing the periodically captured and/or ascertained data, in particular samples, with the initial reference value, in particular threshold value, to identify deviations;
adjusting and/or updating the initial reference value, in particular threshold value, based on the identified deviations, with the value preferably being adjusted and/or updated periodically,
storing the updated reference value, in particular threshold value, as a reference value.

Advantageously, this aspect makes a simple and efficient method possible for comparing the data by way of an automatic adjustment of the reference values and/or threshold values.

According to one or more example embodiments, the item of comparison information comprises an item of system error information. Ascertaining the item of comparison information comprises ascertaining the item of system error information.

The item of system error information comprises, in particular, an item of information which comprises one or more parameter(s) for controlling the imaging apparatus, i.e. in particular items of information or parameters which can determine the mode of operation, the state or the performance of the imaging apparatus. The item of system error information can include, for example, an item of information about spikes, shim (homogeneity of the magnetic field), FatSat (fat saturation) or other, in particular items of error information, items of information of the imaging apparatus which can result in losses in quality, image distortions or measuring errors.

Ascertaining the item of system error information comprises, in particular, measuring, capturing or calculating one more parameter(s) of the imaging apparatus. Ascertaining the item of system error information can comprise, for example, applying one or more tests, sequences, methods or algorithms to the measurement data and/or the expected measurement data in order to determine characteristic values, items of information and/or properties of spikes, shim, FatSat or other system parameters and/or errors. Ascertaining the item of system error information can comprise, in particular, comparing the ascertained parameters with a reference value or threshold value in order to establish whether a system error exists. Ascertaining the item of system error information can comprise, for example, applying a coil reference signal, a shimming process or a fat suppression pulse, in particular to the measurement data, in order to determine parameters such as the homogeneity of the static magnetic field, the sensitivity or linearity of the receive coil or the presence of fat artifacts. The results of these tests or sequences can then be compared, in particular, with the expected measurement data or a reference value or threshold value in order to ascertain the item of system error information.

Advantageously, the item of system error information can be used, in particular, to identify, diagnose or eliminate potential problems, risks or opportunities for improving the imaging apparatus. Ascertaining the item of system error information can enable, in particular, the ascertaining of the cause of system errors and therewith the targeted elimination of them. Therefore the risk of recurring errors, which could result in system failures or unexpected behavior, can be minimized, in addition to optimization of the system performance. The reliability and stability of the imaging apparatus can be guaranteed in the long term by the early identification and correction of system errors.

According to one or more example embodiments, the item of comparison information comprises an item of artifact information. Ascertaining the item of comparison information comprises ascertaining the item of artifact information.

The item of artifact information comprises, in particular, an item of information which quantifies the influence of external or internal sources of interference on the measurement data and/or expected measurement data. The item of artifact information can include, for example, items of information about movement artifacts, breathing artifacts, metal artifacts and/or other factors which can impair the signal quality, the resolution and/or the contrast of the measurement data and/or the imaging.

Ascertaining the item of artifact information comprises measuring, capturing and/or calculating one or more parameter(s) of the imaging apparatus. Ascertaining the item of artifact information can comprise, for example, applying one or more test(s), sequence(s), method(s) or algorithm(s) to the measurement data and/or the expected measurement data in order to determine characteristic values, items of information and/or properties of movement artifacts, breathing artifacts, metal artifacts or other artifact factors. Ascertaining the item of artifact information can also comprise comparing the ascertained items of information with a reference value or threshold value in order to establish whether the measurement data and/or expected measurement data includes artifacts.

The item of artifact information can advantageously be used to evaluate or improve the accuracy or presentability of the measurement data and/or expected measurement data. The causes of artifacts can be precisely identified with the aid of the accurate measurement and analysis of an item of artifact information, such as movement, breathing and metal artifacts. The item of artifact information can enable an initiation of countermeasures to minimize the interference factors, which can in turn result in improved signal quality and greater image contrast.

According to one or more example embodiments, the item of comparison information comprises an item of clinical image information. Ascertaining the item of comparison information comprises a comparison of the expected measurement data and the measurement data with an item of statistical information for ascertaining the clinical image information.

The clinical image information comprises items of information which can preferably be used directly for diagnosis, treatment and/or monitoring of patients, preferably by a doctor. This item of clinical image information can comprise, for example, images or image sequences which clarify the anatomical structure, pathological changes or functional processes in the body of a patient. In contrast to the items of system error information and/or artifact information, in particular no technical deficiencies or interference factors of the imaging apparatus can be mapped via the clinical image information, but rather a medically relevant mapping and/or information of the measurement data and/or expected measurement data can be provided.

The clinical image information can advantageously be used to make precise diagnoses and support therapeutic decisions, while items of system error information and artifact information tends to serve for optimization and quality control of the imaging process. The clinical image information can, in particular, enable accurate mapping and interpretation of the patient data. This can enable a precise diagnosis of diseases and support of therapeutic decisions. The comparison of clinical image information with expected measurement data makes it possible for radiologists and doctors to identify deviations and anomalies more easily and thus make informed decisions.

According to one or more example embodiments, the method comprises a statistical evaluation of the expected measurement data and/or the measurement data for determining an item of evaluation information. The method additionally comprises an updating of statistical information on the basis of the item of evaluation information. The statistical information is stored in a database.

The statistical information comprises one or more item(s) of statistical information. In particular, the statistical information can comprise empirical values and/or empirically ascertained values. For example, the statistical information can comprise fundamental, in particular average, characteristic values, such as a mean, median for, by way of example, parameters and/or medical evidence and/or the results of an application of an statistical inference method, such as a hypothesis test and a regression analysis, to a reference imaging. The statistical information is preferably stored in a database. In particular, the statistical information can comprise reference values and/or threshold values.

According to one or more example embodiments, ascertaining an item of comparison information comprises a statistical evaluation of the measurement data and expected data, at least comprising one of the following steps:
ascertaining a characteristic value,
applying an statistical interference method, with the statistical interference method comprising, in particular, a hypothesis test, a regression analysis and/or a sample analysis,
applying a variance analysis
validating the measurement data and/or expected measurement data via a cross-validation and/or bootstrapping.

The statistical evaluation of the measurement data and/or expected measurement data (referred to together as data) can comprise, in particular, a plurality of optional steps. The data and/or items of information can be subdivided, in particular into groups of data and/or items of information. One group of data and/or items of information can comprise, for example, a plurality of imaging parameters.

Firstly, in particular the data can be described, in which description fundamental statistical characteristic values, in particular, such as a mean, a median, a standard deviation and a variance can be ascertained. These characteristic values can advantageously be used to evaluate and/or map the distribution and/or scattering of the data.

In addition statistical interference methods, in particular, can be applied to identify differences and/or correlations between the data, in particular the measurement data and expected measurement data. Statistical interference methods comprise, in particular, methods and/or processes which are embodied for generating an item of information for a totality (also population) of data starting from a sample. In other words, statistical interference methods enable, in particular, estimation of an item of information, in particular a population parameter, via samples from the data. In particular, applying a probability model can additionally be incorporated in order to quantify the uncertainty, in particular, of these estimations. In particular, determining a confidence interval can be incorporated, which indicates a range of values in which the information, in particular the population parameter, lies with a specific probability.

The statistical interference methods can comprise, in particular, a hypothesis test, such as a t-test or Chi-square test. In particular, the statistically significant differences between groups of items of information and/or data can be ascertained by a hypothesis test. A typical hypothesis test is, in particular, the t-test which is used to establish whether a mean of a sample deviates significantly from a known or assumed population mean. The Chi-square test, by contrast, can be used, in particular, for checking correlations between two categorial, in particular dichotomous, items of information. In particular the correlations, in particular dependencies, between items of information can be qualified and quantified via the regression analysis. Hypothesis tests can advantageously be used to check a proposed hypothesis in that the probability with which the observed data would occur when the hypothesis is assumed is calculated. Via a variance analysis, the scattering within and/or between the groups of data can to analyze and establish whether the differences are random or systematic.

Finally, the results can, in particular, be validated by cross-validation or bootstrapping techniques. Bootstrapping is typically a statistical method for estimating the distribution of a sample statistic in that samples are taken from the data multiple times with replacement. This method enables the calculation of confidence intervals, standard errors and other measured values of statistical accuracy without parametric assumptions. Bootstrapping is particularly advantageous if the theoretical distribution of a statistic is not known or if the sample size is small. Repeatedly taking samples and recalculating the desired statistical bootstrapping enables a robust estimation of the variability and reliability of the items of information from the original sample. Typically an algorithm is used which automates the sampling in a plurality of iterations and generates the distributions of the items of information (statistics) as well as their confidence intervals. Via these methods it is advantageously possible to check the item of comparison information for robustness, generalizability, reliability and/or reproducibility.

The item of evaluation information preferably comprises a result of the statistical evaluation of the measured and expected data. It comprises, for example, statistical characteristic values, such as a mean and/or the results of statistical interference methods such as hypothesis tests and regression analyses. The item of evaluation information can be embodied to be compared with the statistical information. The statistical information can be updated on the basis of the item of evaluation information, in particular via a trained function.

The determination and updating of the statistical information advantageously enables a comparison value and/or reference value which is always current. The current/updated statistical information can enable a comparison of the measurement data and/or expected measurement data with an empirically ascertained value and provide information for improving imaging and/or diagnosis. In addition, the statistical information can be used to carry out more precise and more reliable analyses. In particular in the field of medical diagnostics, it is possible to advantageously use a comparison value and/or an item of comparison information in the form of an item of statistical information, for example to evaluate an accuracy and/or reproducibility.

According to one or more example embodiments, the method additionally comprises:
generating a representation of the item of comparison information for display to a user in a user interface. The representation optionally comprises one or more reaction option(s). Generating the representation optionally comprises creating at least one reaction option from the item of comparison information.
providing the representation to the user in the user interface
receiving, optionally via the user interface, a user input which is aimed at selecting a reaction option
adjusting the item of output information based on the user input
providing the item of output information.

The user interface can be provided, in particular, by a front end computing facility. The user interface can be incorporated, in particular, by a medical imaging apparatus. In other words, the user interface can be, in particular, a user interface of the medical imaging apparatus. The user interface can be embodied, in particular, as a processing position or processing station at which a user (in particular a medical member of staff, such as a female doctor) can retrieve and/or view and/or analyze datasets, in particular the item of comparison information or the representation of the item of comparison information and/or a reaction option and/or at which the user can retrieve and/or view and/or modify the item of output information. The front end computing facility and/or the user interface can have a user interface for this. The front end computing facility and/or user interface can be embodied, in particular, as what is known as a client.

According to one aspect, in the step of providing representation to the user in the user interface, the user interface is provided with the item of comparison information for further processing by a user. According to some examples, for further support the user can additionally be provided with an item of reference information or parts of it, the item of output information and/or one or more items of reaction information.

According to one aspect, the method also comprises a step of receiving a user input of the user, directed at a reaction option, via user surface, in particular a user interface. At least one reaction option can be created from the item of comparison information, in particular via an analysis function. The analysis function [is] also embodied, in particular, to select one reaction option, when a plurality of reaction options have been created, additionally and/or in particular based on a directed user input, and in the step of adjusting the item of output information via the selected reaction option, the item of output information. The item of output information and/or the reaction option can be adjusted, in particular automatically, via a computing unit, in particular the user interface. The reaction function can comprise, for example, an adjusted and/or pre-allocated measurement protocol and/or an indication of an elimination of a system error due to a change in a parameter. For example, the user can be provided with two sets of parameters for selection, which comprise markings and/or values and/or values ranges for changes in parameters, from which the user can select one set of parameters.

According to one aspect, the user input preferably comprises a detail and/or one or more reaction option(s) by the user. Such a user input can comprise a selection of one or more reaction option(s) from a predetermined set (or a list) of reaction options. In particular, a reaction options can be created on the basis of predetermined items of reference information. For example, an analysis function can be embodied to compare the item of comparison information with the items of reference information in order to thus ascertain one or more suitable reaction option(s). The analysis function can be embodied, for example, to ascertain and/or update such reference information. The user input can additionally comprise, in particular, an input of changes in the items of output information, for example the input of a measuring protocol parameter, and/or processing of the item of comparison information, for example a marking of an item of comparison information or selected items of partial information of the item of comparison information.

According to one aspect, the user input can additionally be directed at one or more of the following input(s):
defining a parameter, in particular of the item of output information,
detection of a (medical) abnormality in the items of comparison information,
creating a reaction option of an abnormality shown in the items of comparison information,
selecting an analysis tool for creating a reaction option of an abnormality shown in the items of comparison information, and/or
setting one or more playback parameter(s) for representation of a dataset, in particular the item of comparison information and/or item of output information, on the user surface, in particular in the user interface.

The items of output information can be adjusted in a more targeted manner, or the reaction options can be selected in a more targeted manner by taking the user input into account. The user interface advantageously enables the user to analyze and adjust information efficiently. This results in more precise and individually tailored results, in particular of the item of output information. In particular, the user can react directly to specific anomalies due to the creation and selection of reaction options based on the item of comparison information. This improves the accuracy and rapidity of the adjustment of the items of output information and therewith of the medical imaging. Overall, the user is thereby effectively supported in the expedient and efficient processing of the items of output information, in particular for a subsequent medical imaging examination.

According to one or more example embodiments, the method additionally comprises:
adjusting the item of output information based on the item of comparison information. The item of imaging information of the item of output information comprises a measurement protocol. Adjusting the item of output information comprises adjusting the measurement protocol for a subsequent medical imaging examination.
providing the item of output information to the medical imaging apparatus in order to carry out the subsequent medical imaging examination.

The item of output information, in particular the measurement protocol, can be adjusted by way of a comparison of the item of comparison information with a predetermined items of reference information. The adjustment and/or modification of the items of output information can in particular automatically and, in particular, on the basis of the items of reference information in order to optimize a pending imaging examination. During the adjustment step the item of comparison information can preferably be analyzed and evaluated in order to identify and implement (necessary) changes to the measurement protocol. In particular, the items of output information can be adjusted automatically and without direct user input. A trained analysis function can be applied here. The trained analysis function can be embodied and/or trained to autonomously evaluate the item of comparison information and derive suitable adjustment measures. The analysis function can use, in particular, algorithms and machine learning methods to ascertain patterns and anomalies in the items of comparison information and to update the items of output information on this basis.

The automatic adjustment can take place, in particular, in a plurality of steps:
capturing the items of comparison information,
identifying adjustments, in particular based on an analysis of the items of comparison information to identify potential deviations or anomalies
carrying out the adjustments, in particular via an analysis function, which is embodied to modify the items of output information, in particular the measurement protocol, in particular by the identified adjustments,
validation of the adjustments and/or items of output information.

Advantageously, a targeted and, in particular, automatic adjustment of the item of output information is made possible. For example, the imaging can be optimized by adjusting the parameters of the item of output information, which are decisive for a medical imaging, such as the resolution, the image contrasts and the specific imaging sequences.

According to one or more example embodiments, the method additionally comprises generating a synthetic medical image from the items of output information via an image-generating function. The expected measurement data is generated from the generated synthetic medical image via the trained function.

In particular, the synthetic medical image can be a magnetic resonance image.

The synthetic medical image can comprise, in particular, image data and/or a representation of the examination object, in particular a patient. In particular, the representation of the examination object in the generated synthetic medical image can differ from the representation of a medical image. In particular, the representation of the examination object in the synthetic medical image can at least resemble the representation of the examination object in the medical image or match it. In other words, identical materials, for example, can be represented or mapped in the synthetic medical image and the medical image by identical or similar value ranges of the image data.

In particular, the representation of the examination object in the synthetic image data depends on at least one parameter of the trainable (also trained) function. In other words, the representation of the examination object in the synthetic image data can be specified by the at least one parameter of the trainable function. In particular, the representation of the examination object in the synthetic image data can depend on more than one parameter of the trainable function. The synthetic image data can comprise, in particular, one-dimensional (1D), two-dimensional (2D), three-dimensional (3D) and/or four-dimensional (4D) synthetic image data. The 1D, 2D, 3D and/or 4D synthetic image data can be embodied, in particular, as described above in respect of the 1D, 2D, 3D and/or 4D medical image data. In contrast to the 1D, 2D, 3D and/or 4D medical image data, the 1D, 2D, 3D and/or 4D synthetic image data is generated and/or determined from the output data and not captured on the basis of a medical imaging.

The image-generating function can comprise, in particular, a trained function embodied for image generation. In particular, the image-generating function be embodied to generate a synthetic image from items of output information. The image can resemble and/or match, in particular, a medical image reconstructed from measurement data of a medical imaging examination. The image-generating function can comprise different algorithms and methods in order to process the captured items of output information and transfer them into a visually interpretable image and/or data format. The image-generating function can comprise parameters via which the properties, such as a resolution, of the generated image can be set. In particular, the synthetic medical image can be generated on a remote server and/or a server with greater computing power than that of a computing unit of a medical imaging apparatus.

The generated images advantageously preferably have a high image quality since these images have already been optimized and refined by different algorithms. This can result in more precise expected measurement data. Raw data is often extensive and requires a considerable amount of storage space. Generated images, by contrast, are already processed and compressed, and this significantly reduces the volume of data. This advantageously facilitates storage and fast access to the data. Since generated images have already been pre-processed, the expected measurement data derived therefrom can be calculated and analyzed more quickly. The modification of different parameters, such as resolution and image quality, enables a specific adjustment of the generated images to the respective requirements of the imaging examination. The computing power of the imaging apparatus and/or systems can be used more efficiently due to the use of generated images. The imaging apparatus and/or systems do not have to process the large and often complex raw data, but can access the optimized generated images directly. This results in more efficient use of the existing hardware resources and improved performance of the imaging apparatus and/or systems.

Additionally proposed according to one or more example embodiments is a computing unit for providing an item of comparison information on the basis of items of output information according to one of the preceding described aspects.

Preferably, components of example embodiments, in particular the computing unit, are in the form of a “Cloud service”. Preferably, the computing system of the Cloud, the network, as well as the medical imaging apparatus represent a cluster in the data-technical sense. In particular, the inventive method can be implemented in the network (Cloud) via a command constellation. The data (“result data”) calculated in the Cloud is subsequently sent via the network again to the local computer of the user.

In addition, a medical imaging apparatus comprising a computing unit according to one or more example embodiments is proposed. The computing unit is embodied to control and/or regulate the medical imaging apparatus. The medical imaging apparatus is embodied to carry out a medical imaging examination on the basis of the item of output information and the item of comparison information.

In particular, the medical imaging apparatus can comprise a magnetic resonance apparatus. In particular, a magnetic unit of the magnetic resonance apparatus can comprise a main magnet, a gradient coil unit, a radio-frequency antenna unit. The main magnet of the magnetic unit is preferably embodied to generate a homogeneous (strong, constant) main magnetic field with a defined and/or specific magnetic field strength, such as with a defined and/or specific magnetic field strength of 3 T or 1.5 T. The homogeneous main magnetic field is preferably arranged and/or to be found inside a patient-receiving region of the magnetic resonance apparatus. The magnetic unit conventionally surrounds the patient region (or patient-receiving region) which is embodied to receive a patient for a magnetic resonance examination. The gradient coil unit is preferably embodied to generate gradient fields which are used for spatial encoding during imaging. The radio-frequency antenna unit is preferably permanently arranged inside the magnetic unit and designed and/or embodied to emit an excitation pulse. For capturing the magnetic resonance signals, the magnetic resonance apparatus preferably has local radio-frequency coils which are arranged around the region of the patient to be examined.

The advantages of the proposed computing unit and the proposed medical imaging apparatus substantially match the advantages of the proposed method. Features, advantages or alternative embodiments/aspects of the method can similarly be transferred to other claimed subject matter, and vice versa.

Further, a computer program product is proposed which comprises a program and can be loaded directly into a memory of a programmable system control unit of a medical imaging apparatus and has program means, for example libraries and help functions, in order to execute a proposed method when the computer program product is executed in the system control unit of the medical imaging apparatus. The computer program product can comprise software with a source code, which still has to be compiled and linked or which only has to be interpreted, or an executable software code which for execution only has to be loaded into the system control unit.

The proposed method can advantageously be executed quickly, in an identically repeatable manner and robustly by way of the computer program product. The computer program product is preferably configured such that it can execute the proposed method steps via the system control unit. The system control unit has the requirements respectively, such as an appropriate random-access memory, an appropriate graphics card or an appropriate logic unit, so the respective method steps can be efficiently executed.

The computer program product is saved, for example, on a computer-readable medium or stored on a network or server, from where it can be loaded into the processor of a local system control unit which can be directly connected to the medical imaging apparatus or can be embodied as part of the medical imaging apparatus. Furthermore, items of control information of the computer program product can be saved on an electronically readable data carrier. The items of control information of the electronically readable data carrier can be embodied in such a way that they carry out a proposed method when the data carrier is used in a system control unit of a medical imaging apparatus.

Examples of electronically readable data carriers are a DVD, a magnetic tape or a USB stick, on which electronically readable items of control information, in particular software, is saved. When these items of control information are read from the data carrier and saved in a system control unit of the medical imaging apparatus, it is possible for all proposed embodiments of the method described above to be carried out.

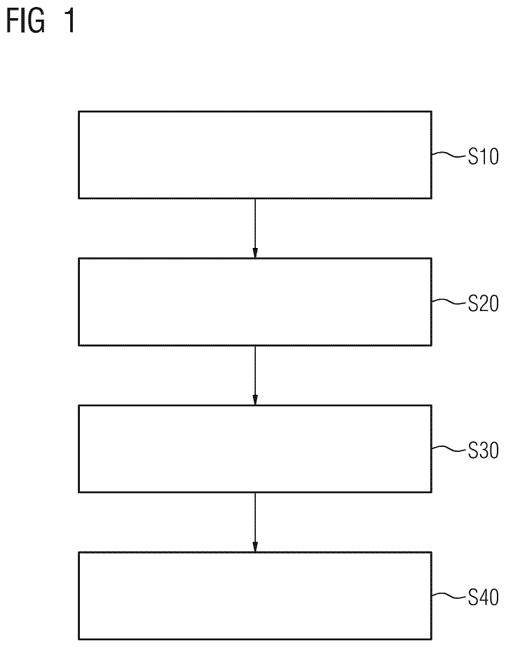

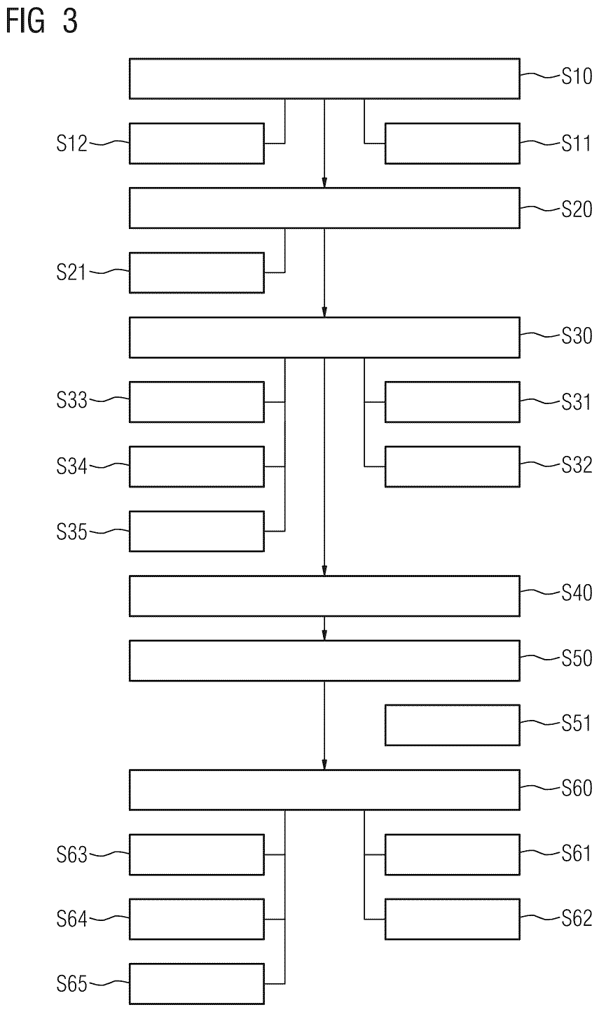

FIG. 1 shows a first exemplary embodiment of a method for providing an item of comparison information BGI on the basis of an item of output information BI. FIG. 1 shows the main method steps of the method in one embodiment, while FIG. 3 additionally illustrates optimum method steps and partial steps of the method in one embodiment.

In a first method step of generating S10 expected measurement data GMD via a trained function TF on the basis of the item of output information BI, the item of output information BI is input into the trained function TF which then generates the expected measurement data GMD. The trained function TF can be, in particular, a deep neural network which has been specifically trained for this. In particular, the neural network can make precise predictions based on medical imaging data (or items of information) and/or patient data (or items of patient information) of the items of output information in the form of expected measurement data. In particular, the trained function can be provided for use on a computing unit.

In a second method step of providing S20 measurement data MD of a medical imaging examination BU on the basis of the items of output information BI, the measurement data MD of a medical imaging examination BU is captured and provided. This measurement data MD comprises detailed items of medical information of the examined patient, in particular in the form of raw data, which can be converted, for example via an image reconstruction method, into medical image data.

In a third method step of ascertaining S30 the item of comparison information BGI on the basis of the expected measurement data GMD and the measurement data MD, the expected measurement data GMD is preferably compared with the actual measurement data MD in order to ascertain the item of comparison information BGI. For example, evaluation functions and or similarity functions can be applied here in order to analyze and quantify the deviations and consistencies between the data.

In a fourth method step of providing S40 the item of comparison information BGI, the ascertained item of comparison information BGI is provided. This item of comparison information BGI can be used to support diagnostic decisions in that it highlights deviations or anomalies in the data. In addition, a measuring and/or imaging protocol can be adjusted, for example, on the basis of the item of comparison information BGI. The provision S40 of the item of comparison information BGI can take place, in particular via an interface, in particular at a medical imaging apparatus. In particular, the item of comparison information BGI can be provided for use on any computing unit, in particular a medical imaging apparatus.

The method steps of generating S10 expected measurement data GMD and of providing S20 measurement data MD of a medical imaging examination BU can also be executed simultaneously or in reverse order. The method steps S10, S20, S30 and S40 can additionally be executed multiple times and/or iteratively and before, after and during the (or a plurality of) imaging examination(s).

The expected measurement data GMD is based, in particular, on a simulated medical imaging of an examination object. The measurement data MD is based, in particular, on a medical imaging of an examination object which has been carried out. The simulated medical imaging can map the medical imaging, which has been carried out, as accurately as possible. However, the simulated medical imaging and the medical imaging which has been carried out can also differ, for example in the imaging modality and/or in a used (assumed) examination protocol. This can be taken into account when ascertaining the item of comparison information BGI. The measurement data MD and expected measurement data GMD can be registered with one another.

The measurement data MD and expected measurement data GMD can comprise raw data and/or medical image data. The medical image data can comprise, in particular, a representation of the examination object. The examination object is, in particular, a patient or, in particular, part of a patient. The examination object can alternatively also be an animal or an article or a part of them/it. For example, the examination object can comprise a thorax of patient. The description of this embodiment will be based below on the assumption, in particular, that the measurement data MD and expected measurement data GMD are present as medical image data.

The medical image data, in particular the measurement data MD and expected measurement data GMD, can comprise a voxel matrix. The voxel matrix can comprise at least one voxel. Alternatively or in addition, the medical image data, in particular the measurement data MD and expected measurement data GMD, can comprise a pixel matrix and/or a time vector. The pixel matrix comprises at least one pixel or the time vector at least one instant. In other words, the medical image data, in particular the measurement data MD and expected measurement data GMD, can be embodied as 1D or 2D or 3D or 4D medical image data. In particular, the voxel matrix and the pixel matrix describe a spatial arrangement of the voxels or pixels. In particular, the time vector describes a course over time of the instants. An image value is associated with each voxel of the voxel matrix. Alternatively, an image value is associated with each pixel of the pixel matrix and/or each instant of the time vector. The mapping of the examination object to these image values describes the representation of the examination object in the corresponding image data.

The representation of the examination object in the medical image data, in particular the measurement data MD and expected measurement data GMD, is specified by the medical modality used and/or by the imaging protocol used. In other words, the medical modality used and/or the imaging protocol specify in which value range the examination object is represented or mapped via the image values. In particular, the representation in respect of the representation of different properties of the examination object can differ. Properties of the examination object are, for example, materials of the examination object and/or potential changes in the examination object.

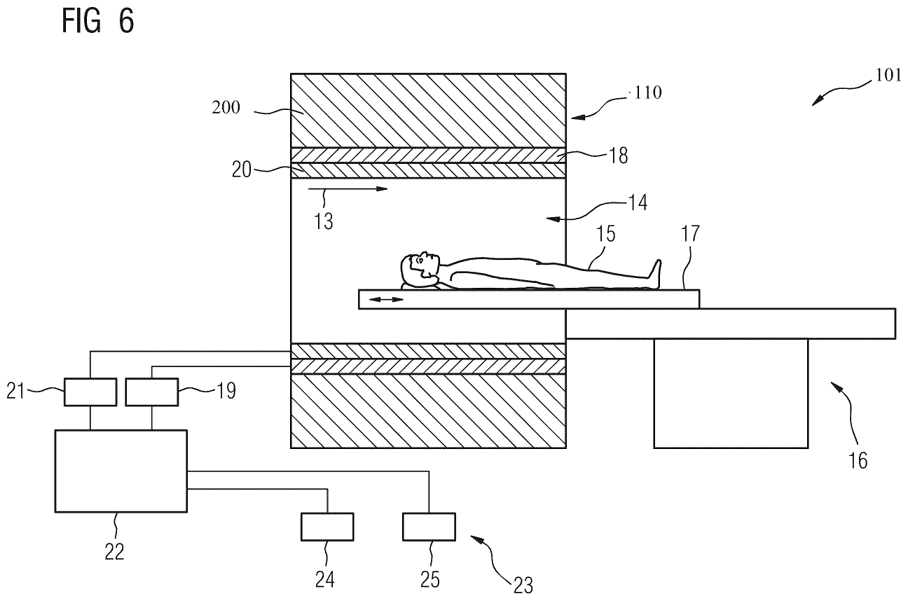

In a preferred exemplary embodiment, the medical image data, in particular the measurement data MD and expected measurement data GMD, is based on magnet resonance tomography (acronym: MRI), in particular with a T1 weighting. In other words, the medical imaging examination is a magnetic resonance imaging, the corresponding medical modality a magnetic resonance imaging apparatus (shown in FIG. 6) and the corresponding imaging protocol a protocol for recording a T1 weighting. Alternatively, the medical image data, in particular the measurement data MD and expected measurement data GMD, can be ascertained on the basis of a computed tomography (acronym: CT). In other words, the medical imaging can also comprise a computed tomography, the corresponding medical modality a computed tomography device (not represented) and the corresponding imaging protocol parameters for recording the medical image data with the computed tomography device.

The medical image data, in particular the measurement data MD and expected measurement data GMD, can be registered with one another. In particular, mutually corresponding regions of the examination object in the medical image data, in particular the measurement data MD and expected measurement data GMD, can be mapped on one another by way of a registration. In particular, a voxel in the expected measurement data GMD can be associated with at least one voxel of the measurement data MD by way of the registration. In particular, a voxel in the expected measurement data GMD can be associated with each voxel in the measurement data MD. In particular, this association is unambiguous. Alternatively, a pixel or instant in the expected measurement data GMD can be associated with at least one pixel or instant in the measurement data MD.

In a method step of generating S10 expected measurement data GMD via the trained function TF on the basis of the item of output information BI, synthetic image data, in particular, can be determined by applying the trainable function to the item of output information BI. In this exemplary embodiment the synthetic image data of expected measurement data GMD comprises a voxel matrix. A voxel of the voxel matrix of the measurement data MD can be associated with each voxel of the voxel matrix of the synthetic image data. In particular, the expected measurement data GMD and the measurement data MD are thus also indirectly registered. Analogously, in alternative exemplary embodiments the synthetic image data can comprise a pixel matrix or a time vector if the measurement data MD comprises a pixel matrix or a time vector. In particular, a pixel or instant in the measurement data MD can then be associated with each pixel or instant of the synthetic image data.

In a method step of ascertaining S30 the item of comparison information BGI on the basis of the expected measurement data GMD and the measurement data MD, a similarity measure, in particular, with a similarity function can be determined. The similarity function compares the expected measurement data GMD and the measurement data MD. The image values of the synthetic image data of the expected measurement data GMD and the medical image data of the measurement data MD are compared. In particular, the image values of those voxels respectively are compared which, according to the registration of the image data of the measurement data MD and expected measurement data GMD, correspond with one another or are associated with one another. The similarity measure depends, for example, on the sum of the squared distances. In particular, the similarity measure depends on the negative sum of the squared distances. Alternatively, the similarity measure can be proportional to the reciprocal of the sum of squared distances. A distance is the discrepancy of the image values from two mutually corresponding voxels (or pixels or instants) in the synthetic image data and in the medical image data. The following applies here: the smaller the sum of the squared distances is, the greater the similarity measure is. In other words, the similarity measure describes how similar the synthetic image data is to the medical image data. Alternatively or in addition, the similarity measure can depend on a cross correlation and/or a normalized cross correlation and/or a covariance and/or a correlation coefficient between the synthetic image data and the medical image data.

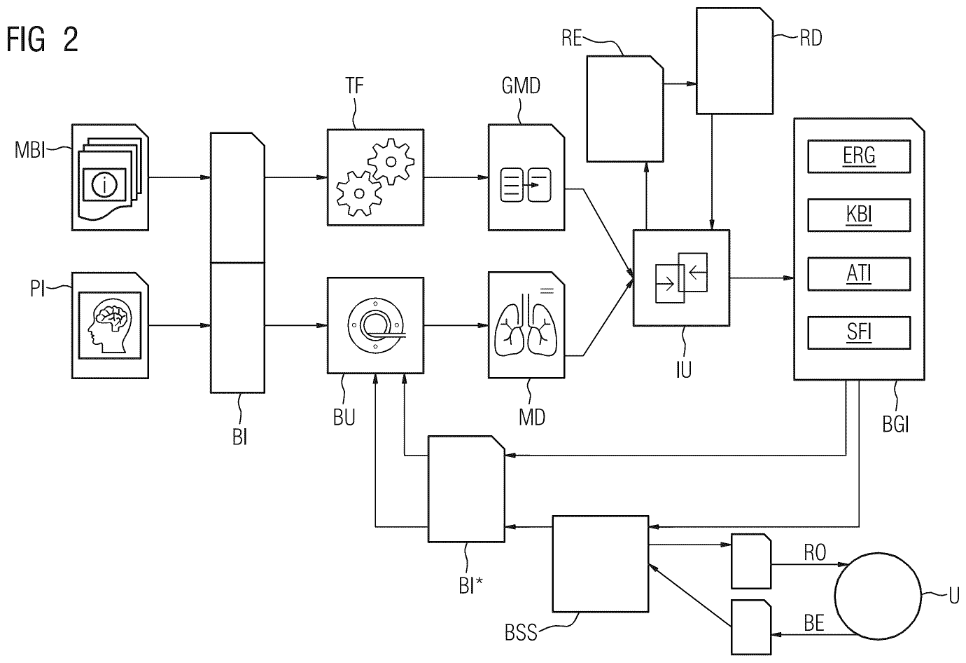

FIG. 2 illustrates on the basis of a data flow diagram the data flow of a method for providing an item of comparison information on the basis of an item of output information according to one embodiment on the basis of a data flow diagram.

The items of output information BI, comprising item of imaging information MBI and items of patient information PI, represent the input data of the imaging examination BU via an imaging apparatus and the trained function TF. The trained function TF generates the expected measurement data GMD. The measurement data MD results from the imaging examination BU. The measurement data MD and expected measurement data GMD are the input data of the comparison unit IU in which the measurement data MD and expected measurement data GMD are compared. The item of output information of the comparison unit IU, the item of comparison information, can comprise an item of system error information SFI, an item of artifact information ATI, an item of outcome information ERG and an item of clinical image information KBI. The comparison unit IU can compare the measurement data MD and expected measurement data GMD via a reference value RD. In addition, the reference value RD can be updated and/or assigned, preferably in a database, via an item of evaluation information from the comparison of the measurement data MD and expected measurement data GMD. The item of output information BI can be adjusted and/or modified via the item of comparison information BGI. The adjusted item of output information BI* can be used as a new item of input information for an imaging examination BU. In addition, the item of comparison information BGI can be displayed to a user U and/or be processed via an user interface BSS. For this purpose, the user interface can be embodied to generate one (or more) reaction option(s) and display them to the user U. The user can make a user input BE via the user interface. The user input BE can be processed by the user interface BSS or the item of output information BI can be adjusted and/or modified via the user input BE via the user interface BSS.

In addition to the method steps S10, S20, S30 and S40 shown and described in FIG. 1, FIG. 3 shows the optional further method steps S50, S60, as well as possible, optional partial, sub-steps of the respective method steps S10 to S60. For example, method steps S11 and S12 are, in particular, optional method steps of the method step S10 and/or are incorporated by it. This applies accordingly to the method steps S31-S35 and S30, S21 and S20, S51 and S50, S61-S65 and S60. The optional method steps can be executed in order, for example step S31, S32, S33, or in any order. The expected measurement data EMD and measurement data MD can be referred to and/or combined as data and/or datasets.

In a method step S11, a synthetic medical image can be generated from the items of output information BI via an image-generating function. Generating a synthetic medical image from the items of output information BI includes applying an image-generating function to the items of imaging and patient information MBI, PI of the item of output information. The trained function TF generates expected measurement data EMD which preferably comprises a voxel matrix. This voxel matrix can be synthesized on the basis of the item of output information BI. The trained function can use machine learning to generate precise synthetic image data which can be compared with the actual measurement data MD.

In a method step S12, generating the expected measurement data EMD can comprise executing an image reconstruction method in reverse order. Generating the expected measurement data EMD by way of an image reconstruction method in reverse order comprises the reversal of the image reconstruction algorithms which are normally used for generating clinical and/or medical image data, in particular measurement data MD. Existing image data is led through the inverse reconstruction process in order to generate synthetic data, in particular expected measurement data EMD, which simulate measured data. This method step can be implemented by a trained function.

In a method step S21, providing the measurement data MD of the medical imaging examination can comprise executing the image reconstruction method in forward order. The raw data, which was captured during the imaging, is processed by a fixed sequence of reconstruction algorithms. These algorithms transform the raw data systematically into a clinically usable image format. The process or this method step can comprise a plurality of steps, among them filtering, noise suppression and image segmentation, with the algorithms analyzing and processing the raw and/or image data in a forward order. This enables a graphical representation and/or processing of the examined anatomical and/or pathological structures.

The method step of comparing measurement data MD with the expected measurement data GMD comprises, in particular, a systematic evaluation and analysis of the two datasets via a comparison unit IU. The measurement data MD, which results from the imaging examination, is compared directly with the synthetic data, the expected measurement data EMD, which was generated by a trained function TF. In particular, the data can be compared in terms of values in order to identify and quantify deviations. The item of comparison information BGI, which results from this step, can comprise system error information SFI, artifact information ATI and clinical items of image information KBI.

The method step S32 comprises a comparison of the expected measurement data EMD with the real measurement data MD, taking into account a fixed reference value RD, in particular threshold value. This threshold value serves to identify significant deviations between the two datasets and/or between the data and the threshold value. The comparison unit IU analyzes the datasets, in particular in terms of values, and quantifies the deviations and/or differences. This method step includes the identification and quantification of deviations in that the discrepancies between the expected measurement data EMD and measurement data MD are systematically broken down and evaluated via a threshold value. If a discrepancy between a value or a set of values of the data and this threshold value is detected, then this signalizes a potential inconsistency in the data. The threshold value can be used as a critic limit which determines the sensitivity of the comparison analysis.

The method steps S33, S34, S35 comprise ascertaining the items of information incorporated by the item of comparison information BGI. Method step S33 comprises ascertaining an item of system error information SFI, method step S34 ascertaining an item of artifact information ATI, method step S35 ascertaining an item of clinical image information KBI.

Ascertaining the item of system error information SFI comprises, in particular, calculating and/or comparing (with a reference value) one or more parameter(s) of the item of comparison information BGI, in particular on the basis of the item of imaging information MBI of the item of output information BI. For example, the method step S34 can comprise applying one or more test(s), sequence(s), method(s) or algorithm(s) to the measurement data MD and/or the expected measurement data EMD in order to determine characteristic values, items of information and/or properties of spikes, shim, FatSat or other system parameters and/or errors.

Spikes are typically unexpected and abrupt changes in the signal of an imaging, which typically appear as marked deflections in the measurement data or image data. These can be caused in medical imaging by various factors, such as sudden movements of the patient, electrical disturbances or problems with the imaging software and hardware. Such spikes can introduce artifacts or distortions. In the context of the item of system error information, ascertaining spikes in the measurement data comprises specific tests and algorithms which are aimed at identifying and analyzing these abrupt signal deflections. The characteristic features of the spikes can be established by applying such methods and they can be compared with a reference value or threshold value in order to evaluate their relevance and potential influence on the image quality. Systematic errors can be ascertained from the items of information resulting therefrom.

Shimming is a method which is used in magnetic resonance imaging (MRI) to improve the homogeneity of the static magnetic field. A uniform magnetic field is typically decisive for the quality of the generated images in MRI apparatuses. Non-uniformities in the magnetic field can result in image distortions and artifacts. A shimming step comprises setting and correcting the magnetic field via specific shim coils or ferromagnetic materials which are positioned in the MRI apparatus. These adjustments are made in order to make the magnetic field more uniform and thus optimize the image quality. In the context of the item of system error information, shimming (shim) refers to the identification of non-uniformities in the magnetic field and/or the various factors for disturbances of this kind. The analysis of the item of system error information includes identifying such non-uniformities in order to ascertain systematic errors.

A fat suppression pulse (FatSat) is a method in MRI, which is aimed at suppressing signals of fatty tissue and thus improving the visibility of other types of tissue. Fatty tissue typically emits strong signals in MRI, which can cover the representation of other structures. The use of a fat suppression pulse selectively reduces the signals of fatty tissue, whereby pathological changes, inflammatory processes or anatomical details can be rendered more visible. In the context of the item of system error information, FatSat refers to the identification and analysis or errors or non-uniformities, which can be produced, for example, by the fat suppression method. This method can react to deviations in the magnetic field or to miscalibrations in the MRI apparatus, and thus result in an incomplete suppression of the fat signal or in undesirable image artifacts. Specific tests and algorithms check whether the fat suppression pulse is functioning correctly and whether the images resulting therefrom are free from systematic errors.

Ascertaining the item of artifact information ATI comprises, in particular, capturing movement artifacts, breathing artifacts, metal artifacts or determining other artifact factors. The item of artifact information ATI can be ascertained, in particular, via a reference value and/or threshold value in order to establish whether the measurement data and/or expected measurement data contain artifacts.

The clinical image information KBI comprises items of information which can preferably be used directly for diagnosis, treatment and/or monitoring patients. This clinical image information KBI can comprise, for example, images or image sequences which clarify the anatomical structure, pathological changes or functional processes in the body of a patient. In contrast to the system error information SFI and/or artifact information ATI, in particular no technical deficiencies or interference factors of the imaging apparatus can be mapped via the clinical image information KBI, rather a medically relevant mapping and/or information of the measurement data MD and/or expected measurement data EMD can be provided.

The clinical image information KBI includes data which can be used directly for diagnosis, treatment and monitoring patients. These items of information comprise, for example, images or image sequences which represent the anatomical structure, pathological changes or functional processes in the body of a patient.

Ascertaining the clinical items of image information KBI can comprise, in particular, a segmentation, a registration and/or a classification. The segmentation can comprise, in particular, dividing medical image data into different segments or regions which represent different types of tissue or structures respectively. Segmentation can be carried out semi-automatically or completely automatically. In particular, the segmentation can take place on the basis of a reference value or reference range, and/or a result of the segmentation can be compared with a reference value ore reference region. The registration can comprise a spatial orientation of the data relative to each other. In particular, image data of different (recording) instants, different modalities (for example MRI and CT) and/or types of data (expected measurement data, measurement data, reference data) can be compared by way of a registration. The registration can ensure that the corresponding anatomical structures in the various images are correctly located one above the other. The classification comprises, in particular, the association of segments or features with specific classes, such as healthy tissue, tumor tissue or an inflamed region. Machine learning algorithms and trained models can be used for this, which, based on existing datasets, in particular reference data, learn to identify and classify different types of tissue or pathological changes.

In a method step of adjusting S50 the item of output information, at least one parameter and/or item of information of the item of output information BI can be adjusted, in particular, via a trained (trainable) function, for example by optimizing a similarity function based on the similarity measure. The similarity measure between the synthetic image data (expected measurement data EMD) and the medical image data (measurement data MD) can depend of the at least one parameter and be incorporated by the item of comparison information BGI. The similarity function describes the dependence of the similarity measure on the at least one parameter. In the method step of adjusting S50 the item of output information, in particular at least one parameter of the item of output information BI can be adjusted in such a way that the similarity measure is maximized. In particular, it can be assumed that the expected measurement data EMD does not contain any system errors, artifacts and/or other errors. In particular, the similarity function is optimized by adjusting the at least one parameter in such a way the similarity measure is maximized as a result of the similarity function. In other words, the at least one parameter is adjusted in such a way that by applying the trained function with adjusted parameters, the synthetic image data optimally resembles the medical image data.

Providing the item of output information BI comprises transferring and/or transmitting the, in particular optimized and/or, adjusted item of output information BI* to the imaging apparatus. This means, in particular, that the optimized parameters and items of information, which were ascertained and/or adjusted during the adjusting of the item of output information BI, are transferred to the imaging apparatus.

According to one or more example embodiments, a representation of the item of comparison information BGI can be generated, in particular, via the user interface. The representation can be displayed to a user, in particular via the user interface. In particular, the representation of the item of comparison information can be an item of image information and/or graphical information. In particular, the representation of the item of comparison information BGI can comprise markings and/or notes in order to clarify the items of information of the item of comparison information BGI. For example, the representation of the item of comparison information BGI can comprise a medical image of a region of the body of a patient, which image comprises clinical items of image information KBI relating to the region of the body of the patient, as well as, for example, an item of system error information SFI in the form of a note.

Generating the representation optionally comprises creating at least one reaction option RE from the item of comparison information BGI. Using, in particular, an analysis function, it is possible to ascertain one or more reaction option(s) RE from the items of comparison information BGI, and these can be provided and/or represented preferably in the form of a note and/or a selection.

In a step S62, the user U can be provided with the optional reaction options RE thus created and the representation of the item of comparison information BGI in the user interface. In particular, the representation of the item of comparison information BGI can also be provided to an automated process (for example in the back-end computing facility of the medical imaging apparatus) for further processing in order to adjust the items of output information BI on the basis, in particular, of the reaction options RE.

The optional step S63 provides a human-machine interaction, in particular for selecting a reaction option RE. A plurality of different reaction options RE can thus be provided via the steps S61 and S62, which options can be displayed, for example, in a front end computing facility and/or a user interface. In step S63, a user input BE can then be received which is directed at selecting one or more of the provided reaction option(s) RE and/or discarding others. The selected reaction options RE can then be used to create an adjusted item of output information BI* in step S64.

According to some implementations, it is also possible to report the user inputs BE back to the analysis function in order to improve it - for instance by further training thereof.

The optional step S63 is directed at the inclusion of a user input BE. The user input BE can be input by a user U into the front end computing facility and/or a user interface and be received in a back end computing facility. The user input BE is preferably an input of the user U who makes it in the context of an analysis of the item of comparison information BGI and/or the reaction options RE.

The optional step S64 describes the adjustment of the item of output information BI as a function of the user input BE. In this step, the information input by a user U via a front end computing facility or a user interface is used to modify the previously generated reaction options RE and/or to create the final item of output information BI*. The user input BE enables a targeted selection and discarding of the presented reaction options RE, whereby a personalized and optimized item of output information BI* is produced. The adjustment takes place by the user input BE being reported back to the analysis function which integrates the selected reaction options RE and updates the item of output information BI accordingly.

In a step S65, the final adjusted item of output information BI* can be provided. A user U can be provided with this item of output information BI via a user interface 10, as can a medical imaging apparatus, in particular via appropriate interfaces. The user interface enables the user U to view, analyze and optionally adjust the items of output information BI. This can occur, for example, by way of visual representations on a monitor or by the use of a processing position. Providing the imaging apparatus with the item of output information BI enables an automatic further processing and adjustment of the item of output information BI (according to the imaging modality), so an imaging examination BU can take place based on the provided item of output information BI.

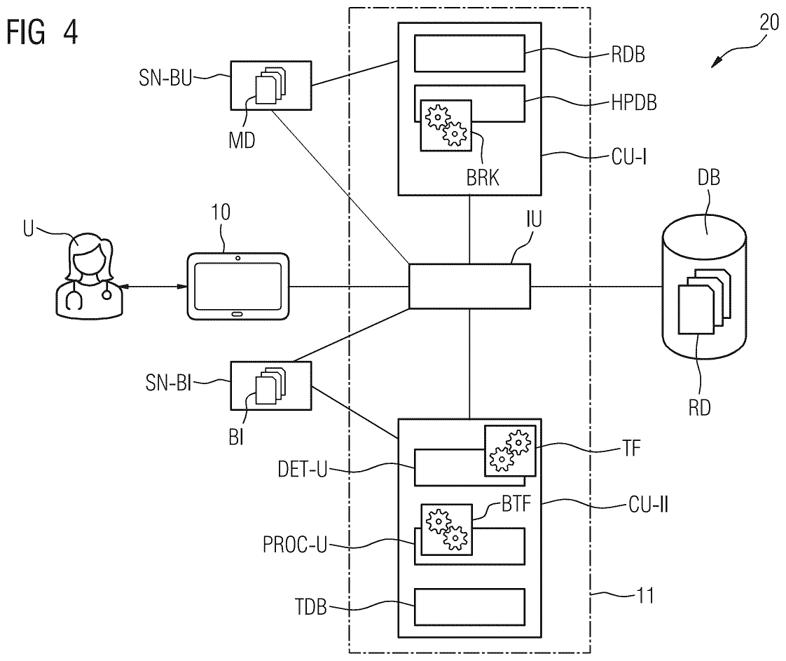

FIG. 4 schematically represents an embodiment of a computing unit 20 for providing an item of comparison information BGI on the basis of items of output information BI. The computing unit 20 has at least one processor CU-I, one processor CU-I, and one comparison unit IU, which together can be referred to as a computing unit 11, in particular and hereinafter as a back end computing unit 11. In addition, a user interface 10 (or front end computing unit 10), a database DB, an interface SN-BU and an interface SN-BI can be incorporated by the computing unit 20 and/or be connected to it.

The back end computing unit 11 and the components: user interface 10, database DB, interface SN-BU and interface SN-BI can have a communications link with each other and/or with a medical imaging apparatus via a medical network or a data interface. The computing unit 20, in particular the back end computing unit 11, can be provided with the item of output information BI, the measurement data MD and further items of information by a storage facility (not represented) and/or the medical imaging apparatus via appropriate interfaces SN-BI, SN-BU. In particular, the components: user interface 10, database DB, interface SN-BU and interface SN-BI and/or back end computing unit 11 can be incorporated by a medical imaging apparatus. In particular, the back end computing unit 11 and/or the components: user interface 10, database DB, interface SN-BU and interface SN-BI can be incorporated by a medical information system and/or network or access them and/or be able to exchange data with the computing facility thereof. In particular, the computing unit 20 and/or the components user interface 10, database DB, interface SN-BU and interface SN-BI, back end computing unit 11 can be part of the same medical organization. A medical organization can be, for example, a practice, a group of practices, a hospital or a group of hospitals. The network connecting these components via the interfaces can accordingly be embodied as an internal network of the organization and comprise, for example, an Intranet (for instance, a Local Area Network and/or a Wireless Local Area Network).

The user interface (also front end computing facility) 10 can be embodied, for example, as a monitor of the medical imaging apparatus or as a processing position, at which a user can view and analyze, for example, the expected measurement data GMD and the measurement data MD as well as create, check, change and assess an item of output information BI. The user interface 10 can have, for instance, a display and/or an input facility for this purpose. The user interface 10 can have a processor. The processor can have a Central Processing Unit (CPU), a Graphics Processing Unit (GPU), a Digital Signal Processor (DSP), an image processing processor, an integrated (digital or analog) circuit or combinations of the components mentioned above and further facilities for providing the expected measurement data GMD, measurement data MD, items of output information BI and/or item of comparison information BGI. The user interface 10 can comprise, for example, a desktop PC, laptop or a tablet.

The user interface 10 can comprise a data retrieval module. The data retrieval module is can be embodied to access the computing unit 20, a medical information system (not represented) and/or a database DB and search for expected measurement data GMD, measurement data MD, items of output information BI and/or item of comparison information BGI, or provide these. In particular, the data retrieval module can be embodied to formulate search queries for an item of information and to parse them to the computing unit 20 and or the database.

The user interface 10 can comprise a user interaction module or unit. This module can be embodied to provide the user with an item of comparison information BGI, an item of output information BI and/or a reaction option RE for further processing. Further, the user interaction module can be embodied to capture one or more user input(s) BE and provide it/them for processing in the back end computing unit 11. Such user inputs BE can comprise, for example, speech, gestures, eye movements, handling of input devices, such as computer mouse, etc. The user inputs BE can be directed toward an interaction with the items of comparison information BGI and/or the items of output information BI, or relate to the creation of adjusted items of output information BI* based on the item of comparison information BGI.

The interfaces SN-BI, SN-BU can generally be embodied to capture and/or save and/or forward datasets. The interface SN-BU can receive and/or provide the measurement data MD. The interface SN-BI can receive and/or provide the items of output information BI. The interfaces SN-BI, SN-BU can comprise a storage unit, in particular a buffer facility.

For example, the computing unit 20 can have a database DB or a plurality of databases (not shown). In particular, the databases can be realized in the Form of one or more Cloud storage modules. Alternatively, the databases can be realized as a local or distributed memory, for example as a PACS (Picture Archiving and Communication System), a hospital information system (KIS) a Labor Information System (LIS), an “Electronic Medical Record” (EMR) information system and/or further medical information systems.

The database DB can be embodied as a central or decentral database. The database DB can be, in particular, part of a server system. The database DB can be, in particular, part of the medical information system. The database DB is embodied, in particular, to save a number of items of reference information RD (or also reference data, comparison datasets). The database DB can also be referred to as a data source, storage facility or storage unit. The database DB can be embodied to provide items of reference information RD and/or other items of information to the computing unit 20, in particular to the comparison unit IU.

The computing unit 20 (also back end computing facility) can have one or more processor(s). Preferably, the computing unit 20 has, as represented, two processors CU-I and CU-II. Further, the computing unit 20 can comprise a comparison unit IU. The processors CU-I, CU-II and/or the comparison unit IU can have a Central Processing Unit (CPU), a Graphics Processing Unit (GPU), a Digital Signal Processor (DSP), an image processing processor, an integrated (digital or analog) circuit or combinations of the components mentioned above and further facilities for providing an item of comparison information BGI according to embodiments. The computing unit 20 can be implemented as individual components or have a group of computers, such as a cluster. Such a system can be called a server system. Depending on the embodiment, the computing unit 20 can also be embodied as a local server and/or constituent part of the medical imaging apparatus. Further, the computing unit 20 can have a main memory, such as a RAM, in order to temporarily save, for example, patient data, data filters and/or image filters, items of information, in particular items of output information BI. The computing unit 20 is embodied, for example, by way of computer-readable instructions, by way of design and/or hardware in such a way that it can execute one or more method step(s) according to embodiments of the present invention.

The comparison unit IU can comprise a module for providing reference data and/or items of information RD. The comparison unit IU can additionally comprise a module (comparison module) for carrying out a similarity analysis. For this, [the] comparison module can be embodied to access the one or more item(s) of reference information and compare it/them with the measurement data and/or expected measurement data. In addition, the comparison module can be embodied to directly compare the measurement data and/or expected measurement data with each other.

The measurement data MD and/or expected measurement data EMD can be distinguished in that they has a certain similarity to each other and/or the items of reference information. For this, [the] comparison module can be embodied, for example, to extract a data descriptor from the measurement data and/or expected measurement data and compare it with corresponding data descriptors from the measurement data, expected measurement data and/or items of reference information. A data descriptor can be understood as a data vector or feature vector, in which relevant features for comparisons of different sets of data and/or information are aggregated. Such relevant features can be extracted from the image data as well as from the non-image data of the datasets. Features extracted from image data can comprise, for example, items of image information, such as patterns, items of color information, intensity values, etc. The data descriptor generated from the measurement data can be compared by the comparison module with corresponding data descriptors of the expected measurement data in order to determine the item of comparison information. For this, the comparison module can ascertain a similarity measure respectively for the items of information being considered, which measure indicates (or quantifies) a similarity of an item of information with the respective associated item of information.

The division, which is carried out, of components of the computing unit 20 into modules serves merely to more simply explain the mode of operation of the computing unit 20 and should not be understood as being limiting. The modules or their functions can also be combined in one and/or more elements. The modules can, in particular, also be understood as computer program products or computer program segments which on execution in the computing unit 20 realize one or more of the described method step(s) described herein.

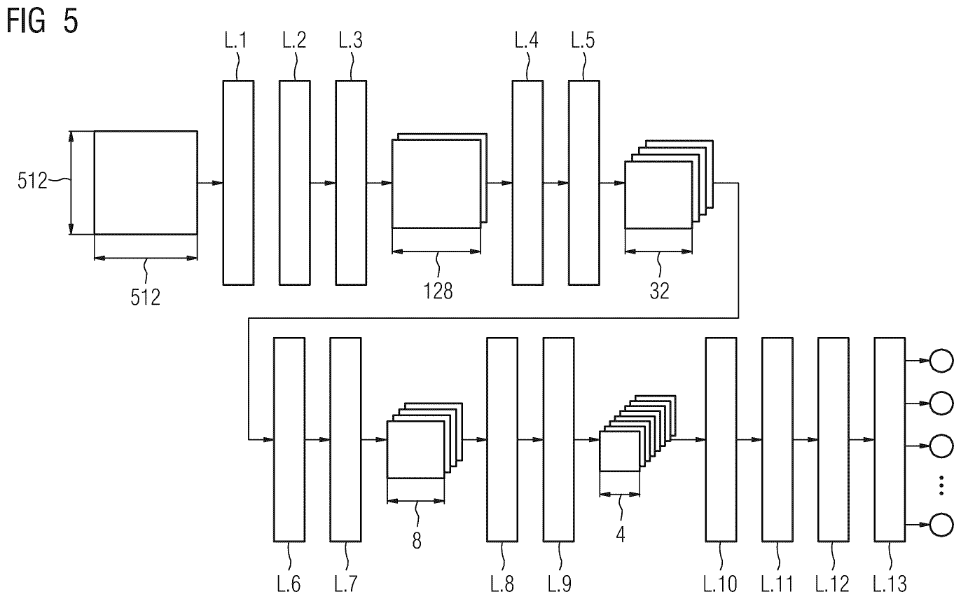

FIG. 5 represents an exemplary embodiment of the trained function TF. In the example shown, the trained function TF is a Convolutional Neural Network (CNN for short), in particular a deep convolutional neural network. As a machine learning model, the trained function TF can process different input data, in particular image data of the expected measurement data GMD and measurement data MD. The trained function TF can process, in particular, pixel or voxel values of a medical image. However, the trained function TF can also process, for example, raw data of the expected measurement data GMD and measurement data MD. However, this is only one illustrative example since the trained function TF can also be embodied by any other suitable model trained by machine learning, such as transformer architectures or FocalNets, as described elsewhere.

The trained function TF according to FIG. 5 comprises convolutional layers, pooling layers and fully connected layers. In the input layer L.1 there is one node for each data element, for example for each pixel of the image data, with each pixel having a channel (the respective intensity value). Four layers L.2, L.4, L.6, L.8 follow the input layer, with each of the four layers being followed by a pooling layer L.3, L.5, L.7, L.9. A 5×5 kernel (indicated by “K: 5×5”) is used for each of the layers, with a padding of 2 (indicated by “P: 2”) and either one or two filter(s)/convolutional kernel(s) (indicated by “F:1” or “F:2”).

Of the pooling layers L.3, L.5, L.7, L.9, the first three layers L.3, L.5, L.7 implement a mean operation over regions o of the size 4×4, and the last pooling layer L.9 implements a maximum operation over regions of the size 2×2. The additional layer L.10 of FIG. 5 smooths the input image data. However, this layer is not relevant to the actual calculation.

The last layers of the network are three fully connected layers L.11, L.12, L.13, with the first fully connected layer having 128 input and 40 output nodes, the second fully connected layer L.12 having 40 input and 10 output nodes and the third fully connected layer L.13 having 10 input and two output nodes, with the two output nodes forming the output layer of the entire machine learning model.

The value of the first node of the output layer can correspond to an element of the item of comparison information BGI (for example, basic parameter of MR or CT image method) of the expected measurement data GMD and measurement data MD, for example one of the medical (comparison) images, which is related to the item of output information BI. The second node can refer to a further element of the item of comparison information BGI (for example, spin echo sequence or gradient echo sequence), etc. There can be as many output nodes present as elements in the item of comparison information BGI which the trained function TF has to differentiate and/or compare.

For training the trained function TF, for example, a database of 500 medical images MI (measurement data MD and expected measurement data EMD) with confirmed items of output information BI and items of comparison information BGI can be used. The database can be divided into training data (320 datasets), validation data (80 datasets) and test data (100 datasets). Then, for example, the image data MI can be extracted from the measurement data MD (and/or expected measurement data GMD). Confirmed items of output information BI can be used for the measurement data MD (and/or expected measurement data GMD). The backpropagation algorithm based on a cost function L(x, y1, y2, . . . yn)=|M(x)1−y1|2+|M(x)2−y2|2+ . . . +|M(x)n−yn|2 can be used for training the trained function TF, where x denotes the input image data, y1 indicates whether a first element of the item of comparison information BGI is displayed, y2 indicates whether a second element of the items of comparison information BGI is displayed, and yn indicates whether an nth element of the item of comparison information BGI is displayed. Furthermore, M(x) denotes the result of applying the trained function TF to the items of output information BI, or the measurement data MD and expected measurement data EMD, and M(x)1, M(x)2, . . . , M(x)n correspond to the values of the first, second, ... nth output node if the trained function TF is applied to the input image data.

Based on the validation set of 80 datasets and the corresponding notes, the best performing trained function TF can be selected from a plurality of machine learning models (with different hyperparameters, for example number of layers, size and number of kernels, padding, etc.). The specificity and the sensitivity can be determined on the basis of the test set of 100 datasets and the items of comparison information BGI.

According to some examples, the medical imaging apparatus can comprise one or more medical imaging modalities, such as a computed tomography system, a magnetic resonance system, an angiography system, C-arm X-ray system, a positron emission tomography system, a mammography system, an X-ray system or the like.

FIG. 6 schematically represents, by way of example, a magnetic resonance apparatus 101. The magnetic resonance apparatus 101 comprises a magnetic unit 110 which has a main magnet 12 for generating a strong and, in particular, time-constant main magnetic field 13. In addition, the magnetic resonance apparatus 101 comprises a patient-receiving region 14 for receiving a patient 15. The patient-receiving region 14 in the present exemplary embodiment is cylindrical and is cylindrically surrounded in a circumferential direction by the magnetic unit 110. However, an embodiment of the patient-receiving region 14 which differs from this is basically conceivable at any time. The patient 15 can be pushed into the patient-receiving region 14 via a patient-supporting apparatus 16 of the magnetic resonance apparatus 101. The patient-supporting apparatus 16 has for this purpose a patient table 17 which is embodied so it can move inside the patient-receiving region 14.

The magnetic unit 110 also has a gradient coil unit 18 for generating gradient fields which are used for spatial encoding during an imaging process. The gradient coil unit 18 is controlled via a gradient control unit 19 of the magnetic resonance apparatus 101. The magnetic unit 110 also comprises a radio-frequency antenna unit 200 which in the present exemplary embodiment is embodied as a body coil permanently integrated in the magnetic resonance apparatus 101. The radio-frequency antenna unit 200 is controlled by a radio-frequency antenna control unit 21 of the magnetic resonance apparatus 101 and irradiates radio-frequency magnetic resonance sequences into an examination space which is substantially formed by a patient-receiving region 14 of the magnetic resonance apparatus 101. The main magnetic field 13 generated by the main magnet 12 consequently establishes an excitation of atomic nuclei. Magnetic resonance signals are generated by relaxation of the excited atomic nuclei. The radio-frequency antenna unit 200 is embodied to receive magnetic resonance signals.

The magnetic resonance apparatus 101 has a system control unit 22 for controlling the main magnet 12, the gradient control unit 19 and the radio-frequency antenna control unit 21. The system control unit 22 controls the magnetic resonance apparatus 101, such as carrying out a predetermined imaging gradient echo sequence. The system control unit 22 can additionally connect the radio-frequency antenna control unit 21 and the gradient control unit 19 and forward control commands to the corresponding control unit. In addition, the system control unit 22 comprises an evaluation unit (not represented) for evaluating the magnetic resonance signals which are captured during the magnetic resonance examination. Furthermore, the magnetic resonance apparatus 101 comprises a user interface 23 which is connected to the system control unit 22. Items of control information, such as imaging parameters, as well as reconstructed magnetic resonance mappings can be displayed on a display unit 24, for example on at least one monitor, of the user interface 23 for a medical member of staff. Furthermore, the user interface 23 has an input unit 25 via which the medical member of staff can input items of information and/or parameters during a measuring procedure.

In conclusion, it will be pointed out once again that the methods described in detail above as well as the represented magnetic resonance apparatus are merely exemplary embodiments which can be modified in a wide variety of ways by a person skilled in the art without departing from the scope of the invention. Furthermore, use of the indefinite article “a” or “an” does not preclude the relevant features from also being present multiple times. Similarly, the term “unit” does not preclude the relevant components from being composed of a plurality of cooperating sub-components which can possibly also be spatially distributed. Independent of the grammatical term usage, individuals with male, female or other gender identities are included within the term.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for providing an item of comparison information based on an item of output information for a medical imaging apparatus, the method comprising:
generating expected measurement data via a trained function based on the item of output information;
providing measurement data of a medical imaging examination based on the item of output information, wherein the item of output information comprises an item of imaging information and an item of patient information;
ascertaining the item of comparison information based on the expected measurement data and the provided measurement data; and
providing the item of comparison information.

2. The computer-implemented method of claim 1, wherein
the generating the expected measurement data comprises executing an image reconstruction method in reverse order, in particular via the trained function, and
the providing the measurement data of the medical imaging examination comprises executing the image reconstruction method in forward order.

3. The computer-implemented method of claim 1, wherein
the ascertaining the item of comparison information comprises comparing the expected measurement data with the provided measurement data, and
the item of comparison information comprises an item of outcome information of the comparison.

4. The computer-implemented method of claim 1, wherein the ascertaining the item of comparison information comprises comparing at least one of the expected measurement data or the provided measurement data with at least one item of reference information.

5. The computer-implemented method of claim 1, wherein
the item of comparison information comprises an item of system error information, and
the ascertaining the item of comparison information comprises ascertaining the item of system error information.

6. The computer-implemented method of claim 1, wherein
the item of comparison information comprises an item of artifact information, and
the ascertaining the item of comparison information comprises ascertaining the item of artifact information.

7. The computer-implemented method of claim 1, wherein
the item of comparison information comprises an item of clinical image information, and
the ascertaining the item of comparison information comprises ascertaining the clinical image information.

8. The computer-implemented method of claim 1, further comprising:
evaluating at least one of the expected measurement data or the provided measurement data for determining an item of evaluation information; and
updating an item of reference information based on the item of evaluation information, wherein the item of reference information is stored in a database.

9. The computer-implemented method of claim 1, further comprising:
generating a representation of the item of comparison information for display to a user in a user interface, wherein the representation comprises one or more reaction options;
providing the representation to the user in the user interface;
receiving, optionally via the user interface, a user input which is aimed at selecting a reaction option;
adjusting the item of output information based on the user input; and
providing the adjusted item of output information.

10. The computer-implemented method of claim 9, further comprising:
adjusting the item of output information based on the item of comparison information, wherein the adjusting the item of output information comprises adjusting the item of imaging information of the item of output information; and
providing the adjusted item of output information to the medical imaging apparatus for carrying out a subsequent medical imaging examination.

11. The computer-implemented method of claim 1, further comprising:
generating a synthetic medical image from the item of output information via an image-generating function, wherein the generating the expected measurement data uses the synthetic medical image.

12. A computing unit configured to provide an item of comparison information using the method of claim 1.

13. A medical imaging apparatus comprising:
a computing unit of claim 12, wherein the computing unit is configured to at least one of control or regulate the medical imaging apparatus, and the medical imaging apparatus is configured to perform a medical imaging examination based on the item of output information and the item of comparison information.

14. A non-transitory computer program product which comprises program components, when executed by a computing unit, cause the computing to perform the method of claim 1.

15. A non-transitory computer storage medium on which program components are stored that, when executed by a computing unit, cause the computing to perform the method of claim 1.

16. The computer-implemented method of claim 2, wherein the generating the expected measurement data executes the image reconstruction method via the trained function.

17. The computer-implemented method of claim 8, wherein the evaluating is statistically evaluating.

18. The computer-implemented method of claim 9, wherein
the generating the representation comprises creating at least one reaction option from the item of comparison information.
